(19) United States
(12) Patent Application Publication
(10) Pub. No.: US 2007/0244394 A1
(43) Pub. Date: Oct. 18, 2007

(54) System and Method of Branch Vessel Marking

(76) Inventor: Trevor Greenan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 11/379,118

(22) Filed: Apr. 18, 2006

(51) Int. Cl. A61B 8/14

(52) U.S. Cl. 600/470

(57) ABSTRACT

A system and method of branch vessel marking including a branch vessel marking system for a branch vessel off a main vessel, the branch vessel having an ostium and a branch vessel centerline, the system having a catheter 105 and a balloon 110 operably attached to the catheter 105. The balloon 110 has a proximal portion 111 and a distal portion 109, a long axis 112, and a coil 114 disposed on the long axis 112. Differential expansion of the distal portion 109 relative to the proximal portion 111 when the distal portion 109 is inflated in the branch vessel and the proximal portion 111 is inflated in the main vessel indicates the ostium of the branch vessel and locates the coil 114 on the branch vessel centerline.

### TECHNICAL FIELD

The technical field of this disclosure is medical navigation devices, particularly, a system and method of branch vessel marking.

### BACKGROUND OF THE INVENTION

Catheters have been developed which include coils to allow electromagnetic tracking of the catheters. The coil location as determined by the electromagnetic tracking can be superimposed on saved fluoroscopic images to indicate the position of the catheter within the body. Unfortunately, catheters cannot always be placed precisely in the body, causing uncertainty in the coil location relative to the body. One particular problem is the location of branching vessels, such as the renal arteries branching off the abdominal aorta. When the catheter tip enters the branch artery from the abdominal aorta, the catheter follows the wall of the branch artery. While this provides a general indication of the location of the branch artery, the indication can vary by the diameter of the branch artery in the worst case. This uncertainty is too large for certain procedures, such as in situ fenestration. In situ fenestration pierces the fabric of a stent graft creating a branch hole to allow blood to flow from the inside of the stent graft into the branch artery. To optimize flow, the center of the branch hole should align with the center of the branch artery. The branch hole cannot be placed precisely when the location of the branch artery is uncertain.

It would be desirable to have a system and method of branch vessel marking that would overcome the above disadvantages.

### SUMMARY OF THE INVENTION

One aspect according to the present invention provides a branch vessel marking system for a branch vessel off a main vessel, the branch vessel having an ostium and a branch vessel centerline, the system having a catheter and a balloon operably attached to the catheter. The balloon has a proximal portion and a distal portion, a long axis, and a coil disposed on the long axis. Differential expansion of the distal portion relative to the proximal portion when the distal portion is inflated in the branch vessel and the proximal portion is inflated in the main vessel indicates the ostium of the branch vessel and locates the coil on the branch vessel centerline.

Another aspect according to the present invention provides a method of marking a branch vessel having an ostium including providing a balloon having a distal end and a long axis, a coil being disposed on the long axis; advancing the distal end into the branch vessel; inflating the balloon to form a proximal portion having a contacting edge; identifying the ostium from the contacting edge; determining whether the coil is within a desired distance of the ostium; and recording a location of the coil with an electromagnetic detection system when the coil is within the desired distance of the ostium.

Another aspect according to the present invention provides a system for marking a branch vessel having an ostium including a balloon having a distal end and a long axis, a coil being disposed on the long axis; means for advancing the distal end into the branch vessel; means for inflating the balloon to form an expanded portion having a contacting edge; means for deducing the ostium from the contacting edge; means for determining whether the coil is within a desired distance of the ostium; and means for recording a location of the coil when the coil is within the desired distance of the ostium.

The foregoing and other features and advantages according to the invention will become further apparent from the following detailed description.

### BRIEF DESCRIPTION OF THE DRAWINGS

FIGS. 1A-1D are side and cross section views of a branch vessel marking system made in accordance with the present invention;

FIG. 2 is a flow chart of a method of branch vessel marking in accordance with the present invention; and

FIGS. 3A-3F are schematic diagrams of a method of branch vessel marking in accordance with the present invention.

### DETAILED DESCRIPTION

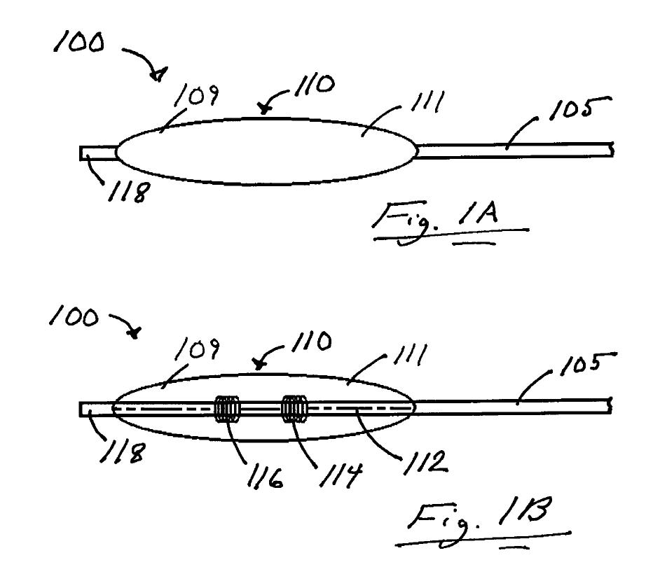

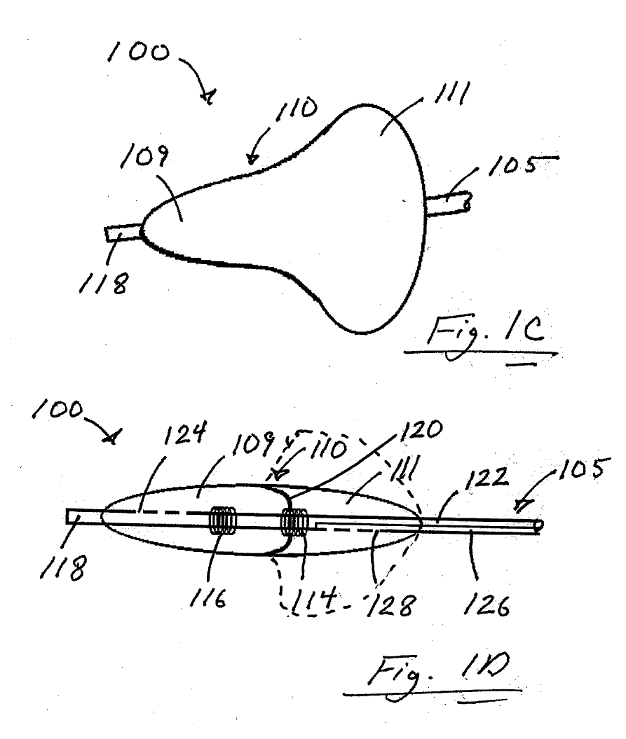

FIGS. 1A-1D, in which like elements share like reference numbers, are side and cross section views of a branch vessel marking system made in accordance with the present invention. Referring to FIG. 1A, which is a side view of a branch vessel marking system, the branch vessel marking system 100 includes a catheter 105 and a balloon 110 operably attached to the catheter 105. The catheter 105 can be any variety of balloon catheters, such as a PTCA (percutaneous transluminal coronary angioplasty) balloon catheter capable of supporting a balloon during angioplasty. In one embodiment, the catheter 105 is a steerable catheter. In one embodiment, the catheter 105 has a distal end 118. The balloon 110 can be manufactured from a material such as polyethylene, polyethylene terephthalate (PET), nylon, Pebax® polyether-block co-polyamide polymers (non-compliant), or polyurethane and silicone (compliant), or the like. The balloon 110 can be inflated with a liquid, such as a contrast medium, through a lumen (not shown) in the catheter 105.

The balloon 110, shown in a partially expanded state, has a distal portion 109 and a proximal portion 111. When the distal portion 109 is inflated in a branch vessel and the proximal portion 111 is inflated in a main vessel, differential expansion of the distal portion 109 relative to the proximal portion 111 indicates the ostium of the branch vessel and locates a coil on the branch vessel centerline. The contacting edge of the balloon 110 where the balloon 110 contacts the main vessel indicates the position of the ostium of the branch vessel. The axial position of the balloon 110 in the branch vessel can be adjusted so that the coil is located at the ostium center when the balloon 110 is inflated, since the intersection of the branch vessel centerline and the ostium defines the ostium center. The location of the coil can be read with an electromagnetic detection system and overlaid onto an indicated ostium position on a live or stored fluoroscopic image to provide navigation guidance during surgery. Those skilled in the art will appreciate that the dimensions of the balloon 110 can be selected for the particular main vessel and branch vessel in which the balloon 110 is to be used.

The balloon 110 can be a uniform balloon, a non-uniform balloon, or a dual balloon. In one embodiment, the balloon 110 is a uniform balloon, i.e., the distal and proximal portions of the balloon are the same compliance. The distal and proximal portions of the uniform balloon are defined by the position of the balloon in the branch vessel. The distal portion is located in the branch vessel and the proximal portion is located in the main vessel. The boundary between the portions changes as the balloon is moved axially in the branch vessel. The uniform balloon has sufficient compliance to allow differential expansion at a low inflation pressure. The differential expansion allows the proximal portion of the balloon 110 positioned outside the branch vessel in the main vessel to expand to a large diameter relative to a distal portion of the balloon 110 positioned inside the branch vessel. Low inflation pressure as defined herein is any inflation pressure above the pressure of the fluid in the vessel capable of expanding the balloon without substantially expanding the branch vessel.

In another embodiment, the balloon 110 is a non-uniform balloon, i.e., the distal and proximal portions of the balloon are different. In one example, the distal portion has one compliance and the proximal portion has another compliance. In another example, the distal portion has one uninflated diameter and the proximal portion has another uninflated diameter. In yet another example, the distal and proximal portions have different compliance and different dimensions.

In another embodiment, the balloon 110 is a dual balloon, i.e., the distal and proximal portions of the balloon are separate balloons. The distal and proximal portions of the balloon can be made of the same materials and have the same dimensions, or can be made of different materials and/or have different dimensions.

Referring to FIG. 1B, which is a cross section view of a branch vessel marking system, the balloon 110 has a long axis 112 coincident with the catheter 105 within the balloon 110 and a coil 114 disposed on the long axis 112. The long axis 112 is illustrated by the dashed center line in FIG. 1B. Electrical leads connecting the coil 114 to an electromagnetic (EM) detection system have been omitted for clarity of illustration (thought they could be tiny). In this example, the balloon 110 is a uniform balloon or a non-uniform balloon.

The coil 114 disposed on the long axis 112 of the balloon 110 can be any variety of coil suited for EM detection as an EM marker. The EM detection system applies an EM field to the patient and detects induced voltages from the coil 114 to identify the location and/or orientation of the coil 114. In one embodiment, the coil 114 also acts as a fluoroscopic marker, visible with fluoroscopy. In another embodiment, a radiopaque marker is located at the coil 114 to improve the fluoroscopic visibility of the coil 114. Those skilled in the art will appreciate that a number of coils can be disposed along the long axis 112 as additional markers. In the example of FIG. 1B, an optional second coil 116 is disposed on the long axis 112 between the first coil 114 and distal end 118.

Referring to FIG. 1C, which is a side view of a branch vessel marking system, the balloon 110 has a distal portion 109 that is smaller than the proximal portion 111, even when the balloon 110 is inflated outside the vessels. The balloon 110 is preshaped to the shape required to indicate the ostium. In this example, the balloon 110 is a non-uniform balloon or a dual balloon. In one embodiment, the coil (not shown) is axially aligned with the transition between the distal portion 109 and the proximal portion 111 to facilitate location of the coil in the ostium center.

Referring to FIG. 1D, which is a cross section view of a branch vessel marking system, the balloon 110 has a distal portion 109 that is one balloon and a proximal portion 111 that is another balloon. In this example, the balloon 110 is a dual balloon. The catheter 105 has a first lumen 122 in communication with the interior of the distal portion 109 through ports 124 and a second lumen 126 in communication with the interior of the proximal portion 111 through ports 128. The distal portion 109 and the proximal portion 111 can be inflated independently through the first lumen 122 and the second lumen 126, respectively (the second balloon taking the shape of a larger diameter when inflated as shown, for example, by the dashed lines). In one embodiment, the coil 114 is located axially at the wall 120 separating the distal portion 109 and the proximal portion 111. In another embodiment, an optional second coil 116 is disposed on the long axis (not shown) between the first coil 114 and the distal end 118.

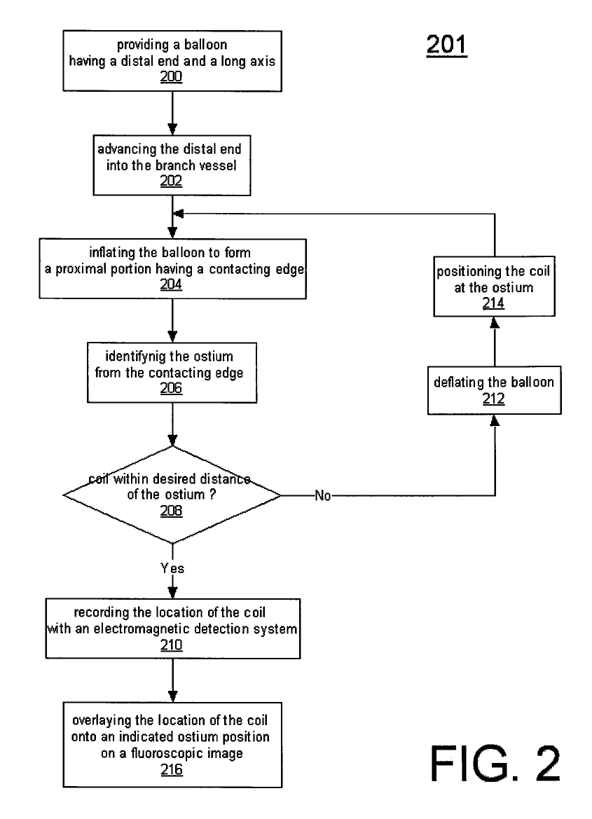

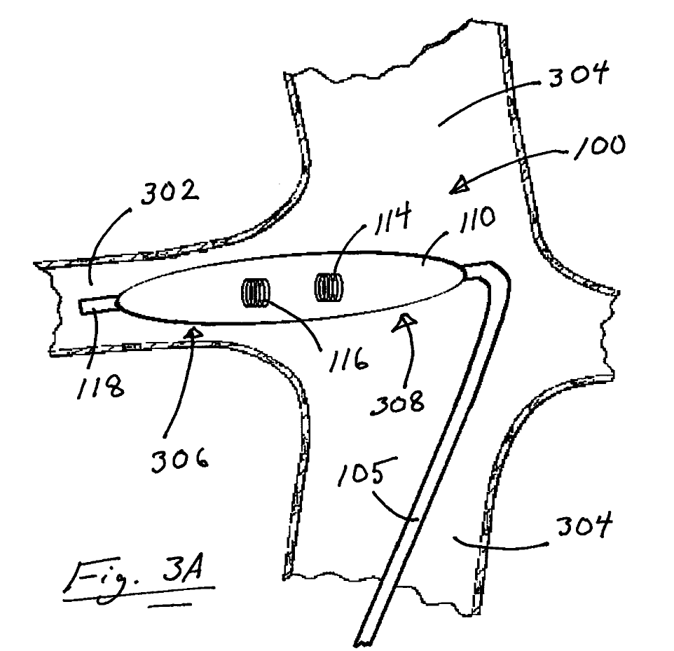

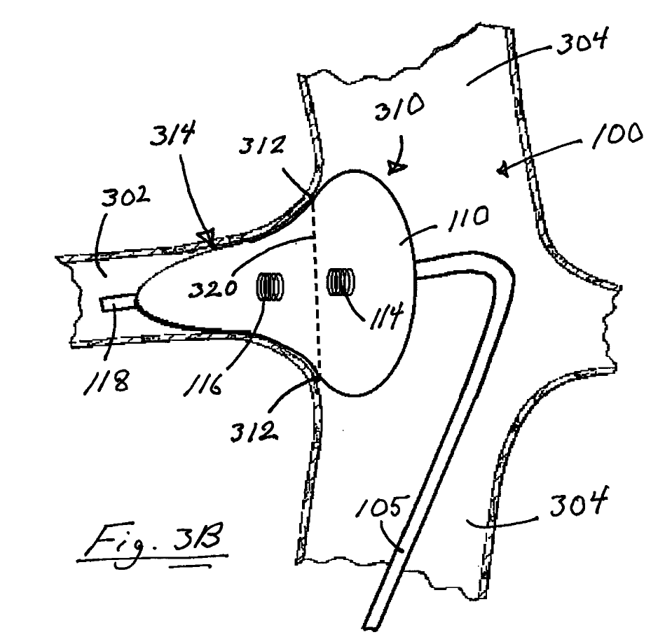

FIG. 2 is a flow chart of a method of branch artery marking in accordance with the present invention. The method 201 includes providing a balloon having a distal end and a long axis with a coil disposed on the long axis 200, advancing the distal end into the branch vessel 202, inflating the balloon to form a proximal portion having a contacting edge 204, identifying the ostium from the contacting edge 206, determining whether the coil is within a desired distance of the ostium 208, and recording the location of the coil with an electromagnetic detection system when the coil is within the desired distance of the ostium 210. When the axial distance is not within the desired distance, the position of the balloon can be adjusted toward the ostium by deflating the balloon 212 and positioning the coil at the ostium 214. The accuracy of the positioning 214 can be checked by reinflating the balloon at 204 and repeating the method 201 until the coil is within the desired distance of the ostium. The balloon centers the coil in the ostium, precisely locating the coil at the ostium center. Once the location of the coil has been recorded with the electromagnetic detection system, the balloon can be deflated and removed from the branch and main vessels as desired. The location of the coil recorded with the electromagnetic detection system can be overlaid onto an indicated ostium position on a live or stored fluoroscopic image 216 to provide navigation guidance during surgery. In one embodiment, the inflating the balloon 204 includes inflating the balloon with a contrast medium and the determining whether the coil is within a desired distance of the ostium 208 includes determining whether the coil is within a desired distance of the ostium visually from a fluoroscopic image.

Overlaying the location of the coil recorded with the electromagnetic detection system onto an indicated ostium position on a live or stored fluoroscopic image registers the ostium center on the live or stored fluoroscopic image. This allows the physician to precisely locate the ostium center during surgery. For example, the distal tip of a catheter can include a coil visible to the physician on a fluoroscopic image and trackable with the electromagnetic detection system. The electromagnetic detection system follows the progress of the distal tip of a catheter and displays its location on the live or stored fluoroscopic images, allowing the physician to visualize the position of the catheter in the vessel. The location of the ostium center is stored in the electromagnetic detection system, so the physician knows precisely when the tip of the catheter is at the ostium center.

Additional information can be obtained when the balloon is at the branch vessel after recording the location of the coil with an electromagnetic detection system, which locates the ostium center for the electromagnetic detection system. In one embodiment, the method continues with deflating the balloon, advancing the distal end further into the branch vessel, inflating the balloon, and recording a second location of the coil with the electromagnetic detection system to locate a branch vessel centerline. This can be repeated to provide a string of coil locations from the repeated recordings to provide the desired length and detail for the branch vessel centerline. When the balloon has a second coil disposed on the long axis between the first coil and the distal end, a second coil location of the second coil can be recorded with the electromagnetic detection system while the first coil is located at the ostium center. The locations of the first and second coils describe the branch vessel centerline.

In another embodiment, the method continues with recording a plurality of coil locations over a time period. The coil remains at the ostium center, so this provides an indication of the ostium center position with time. The method can also record a body function, such as cardiac cycles, respiratory cycles, or the like, over the same time period. The plurality of coil locations can be correlated with the body function to see how the ostium center moves in time with the body function.

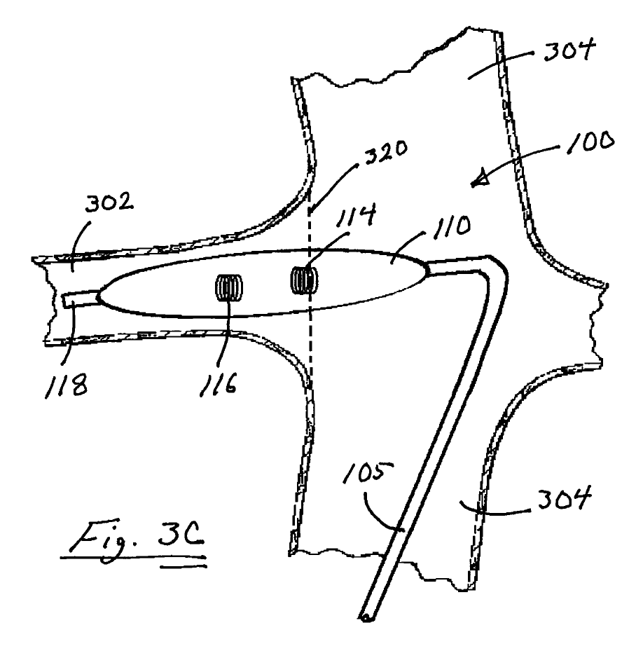

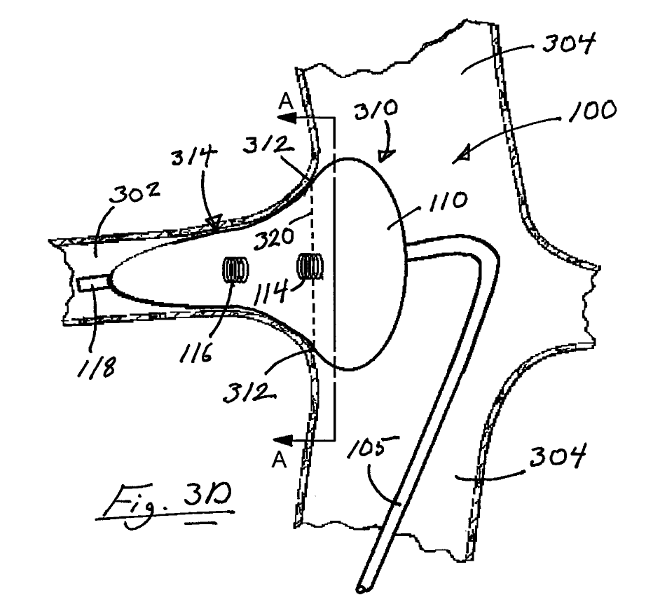

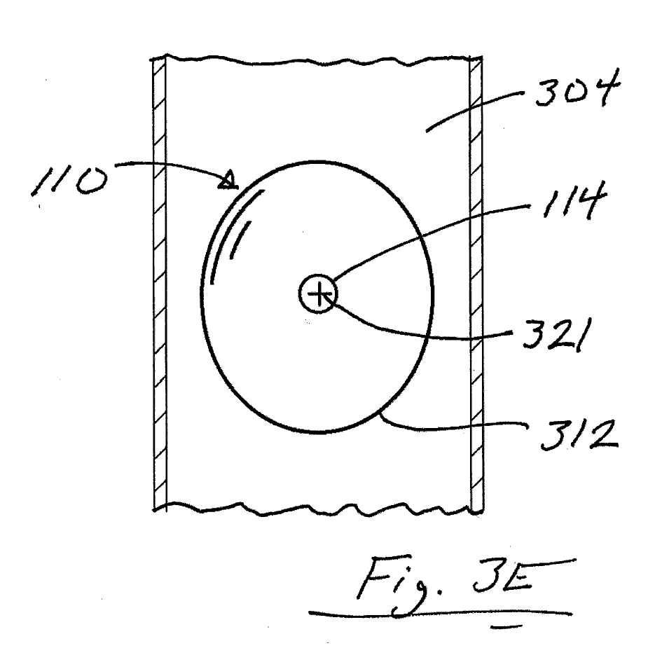

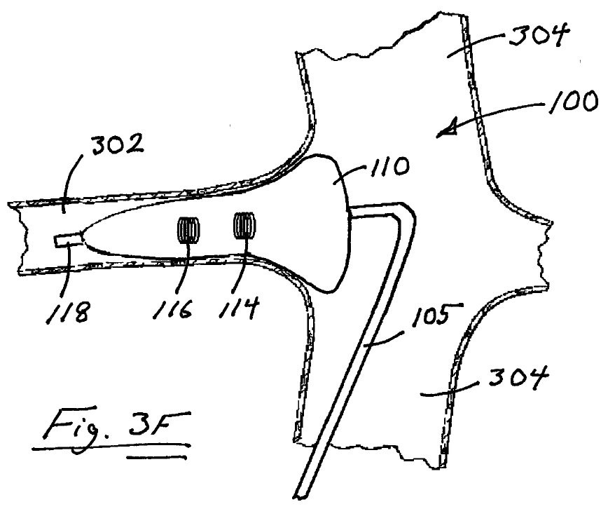

FIGS. 3A-3E, in which like elements share like reference numbers with FIGS. 1A-1B and with each other, are schematic diagrams of a method of branch artery marking in accordance with the present invention.

Referring to FIG. 3A, the distal end 118 of a balloon 110 is advanced into a branch vessel 302 off a main vessel 304, such as one of the renal arteries branching off the abdominal aorta. A distal portion 306 of the balloon 110 is disposed in the branch vessel 302 and the proximal portion 308 is disposed in the main vessel 304. The balloon 110 has a first coil 114 and an optional second coil 116 disposed on its long axis. The catheter 105 can be steerable to direct the balloon 110 into the branch vessel 302. When the balloon 110 is inflated, the distal portion 306 is the portion of the balloon 110 that expands to fill the branch vessel 302 and the proximal portion 308 expands outside of the branch vessel 302.

In one embodiment, a guidewire (not shown) is inserted into the patient and the balloon 110 follows the guidewire through the main vessel 304 into the branch vessel 302. The guidewire can be steerable to direct the guidewire into the branch vessel 302. In another embodiment, a sleeve (not shown) is inserted into the patient and the balloon 110 is inserted in the lumen of the sleeve, following the lumen through the main vessel 304 to the site near the branch vessel 302. In yet another embodiment, a sleeve (not shown) is inserted into the patient, a guidewire (not shown) is inserted through the lumen of the sleeve to the site near the branch vessel 302, and the balloon 110 follows the guidewire into the branch vessel 302. Those skilled in the art will appreciate that the physician performing the procedure will not be able to clearly see the main vessel 304 or the branch vessel 302 on the fluoroscopic image unless a contrast medium is present in the vessels. The physician can see the balloon 110 when the balloon is inflated with a contrast medium and can see the first coil 114 and the optional second coil 116. Further, the anatomy of the main vessel 304 and the branch vessel 302 can vary: the angle of the branch vessel 302 relative to the main vessel 304 depends on the particular patient and is not necessarily the right angle illustrated.

Referring to FIG. 3B, the balloon 110 is inflated to form an expanded portion 310 located within the main vessel 304 and having a contacting edge 312. The contacting edge 312 as defined herein is the generally circular part of the balloon 110 where the balloon 110 intersects the periphery of the ostium 320 when the balloon 110 is inflated in the branch vessel 302. Inflation of the balloon 110 precisely locates the coil 114 along the branch vessel centerline. The balloon 110 also has a constrained portion 314 located within the branch vessel 302. The compliance of the balloon 110 allows differential expansion of the expanded portion 310 relative to constrained portion 314, i.e., the expanded portion 310 becomes larger in diameter than the constrained portion 314 to produce a discernable contacting edge 312. The inflation pressure is low enough that the constrained portion 314 does not significantly expand the branch vessel 302. In one embodiment, the balloon 110 is inflated with a contrast medium so the balloon 110 is visible on a fluoroscopic image. The first coil 114 and second coil 116 are also visible on a fluoroscopic image, either by their construction, by being marked with a radiopaque marker, or by a combination of construction and a radiopaque marker.

The ostium 320 for the branch vessel 302 can be deduced from the contacting edge 312 of the expanded portion 310 of the balloon 110. The dashed line of FIG. 3B illustrates the ostium 320 indicated by the contacting edge 312. The ostium 320 can be deduced by direct observation and/or with computer assistance. It is determined visually or by machine measurement whether the coil 114 is within a desired distance of the ostium 320. Those skilled in the art will appreciate that the measurement of the desired distance can be performed by various computer assisted methods and that the desired distance can be selected depending on the accuracy required for a particular procedure. Should the coil 114 be within the desired distance of the ostium 320, the location of the coil 114 can be recorded with an electromagnetic detection system. In the example of FIG. 3B, the coil 114 is not within the desired distance of the ostium 320. Referring to FIG. 3C, the balloon 110 is deflated and the coil 114 is positioned at the ostium 320. Because the balloon 110 is deflated and lacks a contacting edge, there is no observable indication of the ostium 320 on the fluoroscopic image. The positioning of the coil 114 at the ostium 320 is estimated using the distance between the coil 114 and the ostium 320 observed when the balloon 110 was last inflated.

Aligning the coil 114 with the ostium 320 precisely centers the coil 114 at the ostium center when the balloon 110 is inflated. The desired distance allowed between the coil 114 and the ostium 320 determines the accuracy with which the ostium center is located when the position of the coil 114 is recorded with the electromagnetic detection system.

Referring to FIG. 3D, the balloon 110 is reinflated to check the accuracy of the positioning of the coil 114 at the ostium 320. The ostium 320 is deduced from the contacting edge 312 of the expanded portion 310 of the balloon 110. The deflating, positioning, and reinflating of the balloon can be repeated until the coil 114 is within the desired distance of the ostium 320. In the example of FIG. 3D, the coil 114 is within the desired distance of the ostium 320, so the location of the coil 114 is recorded with an electromagnetic detection system. The inflation of the balloon 110 and location of the coil 114 on the long axis of the balloon 110 assure that the coil 114 is located along the branch vessel centerline. Because the coil 114 is also aligned with the ostium 320, the coil 114 is precisely located at the ostium center.

The precise location of the coil 114 with the electromagnetic detection system can be overlaid onto the indicated ostium position on stored fluoroscopic images to assure precise registration between future electromagnetic detection system measurements and the stored fluoroscopic images. This can be used to provide precise navigation guidance during surgery, such as in situ fenestration of a stent graft in an abdominal aorta. In situ fenestration creates a hole in the fabric of the stent graft to allow blood to flow from the inside of the stent graft into the renal artery. Precise navigation allows the physician to create the hole at the ostium center. Once the location of the coil 114 has been recorded with the electromagnetic detection system, the balloon 110 can be deflated and removed from the branch and main vessels as desired.

Referring to FIG. 3E, which is the view along section A-A of FIG. 3D, the coil 114 is precisely located at the ostium center 321. The catheter supporting the coil 114 is omitted for clarity of illustration. The contacting edge 312 is located at the periphery of the ostium of the branch vessel.

If desired, the branch vessel marking system 100 can be used to obtain additional information on the branch vessel before the balloon is removed from the branch vessel. Referring to FIG. 3D, the balloon 110 can have an optional second coil 116 disposed on the long axis of the balloon 110 between the first coil 114 and the distal end 118. A second coil location of the second coil 116 can be recorded with the electromagnetic detection system while the balloon 110 is inflated and the first coil 114 is located at the ostium 320. The location of the first coil 114 and the second coil 116 describes the branch vessel centerline since the inflation of the balloon 110 and location of the coils 114, 116 on the long axis of the balloon 110 assure that the coils 114, 116 are located at the center of the branch vessel 302.

A plurality of coil locations can be recorded with the electromagnetic detection system over a time period while the balloon 110 is inflated and the first coil 114 is located at the ostium 320. This provides an indication of ostium 320 with time. One or more body functions, such as cardiac cycles, respiratory cycles, or the like, can also be recorded over the same time period. The plurality of coil locations can be correlated with the body function to see how the ostium 320 moves in time with the body function.

Referring to FIG. 3F, the balloon 110 has been deflated, the distal end 118 has been advanced further into the branch vessel 302 relative to the position illustrated in FIG. 3D, and the balloon 110 has been reinflated. A second location of the coil 114 as illustrated in FIG. 3F can be recorded with the electromagnetic detection system and combined with the previously obtained location when the coil 114 was at the ostium 320 as illustrated in FIG. 3D to locate a branch vessel centerline. Those skilled in the art will appreciate that the anatomy of the main vessel 304 and the branch vessel 302 can vary: the angle of the branch vessel 302 relative to the main vessel 304 depends on the particular patient and is not necessarily the right angle illustrated. A string of coil locations can be obtained from repeatedly recording the coil location and advancing the balloon 110 to provide the desired length and detail for the branch vessel centerline and the angle of the branch vessel centerline relative to the main vessel 304. Greater accuracy and/or greater recording speed can be obtained when the balloon 110 includes a number of coils, so that a number of centerline positions can be determined at each balloon position. Once all the desired information on the branch vessel has been recorded, the balloon 110 can be deflated and removed from the branch and main vessels.

While specific embodiments of the invention are disclosed herein, various changes and modifications can be made without departing from the spirit and scope of the invention. The scope of the invention is indicated in the appended claims, and all changes that come within the meaning and range of equivalents are intended to be embraced therein.

1. A branch vessel marking system for a branch vessel off a main vessel, the branch vessel having an ostium and a branch vessel centerline, comprising:
a catheter 105; and
a balloon 110 operably attached to the catheter 105, the balloon 110 having a proximal portion 111 and a distal portion 109, a long axis 112, and a coil 114 disposed on the long axis 112;
wherein differential expansion of the distal portion 109 relative to the proximal portion 111 when the distal portion 109 is inflated in the branch vessel and the proximal portion 111 is inflated in the main vessel indicates the ostium of the branch vessel and locates the coil 114 on the branch vessel centerline.

2. The system of claim 1 wherein the differential expansion occurs at a low inflation pressure.

3. The system of claim 1 wherein the balloon is selected from the group consisting of a uniform balloon, a non-uniform balloon, and a dual balloon.

4. The system of claim 1 further comprising a radiopaque marker located at the coil.

5. The system of claim 1 wherein the balloon 110 has a distal end 118 and the coil 114 is a first coil, further comprising a second coil 116 disposed on the long axis 112 between the first coil and the distal end 118.

6. The system of claim 1 wherein the balloon 110 is made of a material selected from the group consisting of polyethylene, polyethylene terephthalate (PET), nylon, and polyether-block co-polyamide polymers.

7. A method of marking a branch vessel having an ostium comprising:
providing a balloon having a distal end and a long axis, a coil being disposed on the long axis 200;
advancing the distal end into the branch vessel 202;
inflating the balloon to form a proximal portion having a contacting edge 204;
identifying the ostium from the contacting edge 206;
determining whether the coil is within a desired distance of the ostium 208; and
recording a location of the coil with an electromagnetic detection system when the coil is within the desired distance of the ostium 210.

8. The method of claim 7 further comprising:
deflating the balloon when the coil is not within the desired distance of the ostium 212; and
positioning the coil at the ostium 214.

9. The method of claim 7 wherein the inflating comprises inflating the balloon with a low inflation pressure.

10. The method of claim 7 further comprising correlating the location of the coil recorded with the electromagnetic detection system and an indicated ostium position on a fluoroscopic image 216.

11. The method of claim 7 further comprising:
deflating the balloon;
advancing the distal end further into the branch vessel;
inflating the balloon; and
recording a second location of the coil with the electromagnetic detection system to locate a branch vessel centerline.

12. The method of claim 7 further comprising recording a plurality of coil locations over a time period.

13. The method of claim 12 further comprising:
recording a body function over the time period; and
correlating the plurality of coil locations with the body function;

14. The method of claim 13 wherein the body function is selected from the group consisting of cardiac cycles and respiratory cycles.

15. The method of claim 7 wherein the inflating comprises inflating the balloon with a contrast medium, and the determining whether the coil is within a desired distance of the ostium 208 comprises determining whether the coil is within the desired distance of the ostium visually from a fluoroscopic image.

16. The method of claim 7 wherein the branch vessel is a renal artery and the main vessel is an abdominal aorta.

17. The method of claim 7 wherein the coil is a first coil, and the balloon further comprises a second coil disposed on the long axis between the first coil and the distal end, further comprising:
recording a second coil location of the second coil with the electromagnetic detection system to locate a branch vessel centerline.

18. A system for marking a branch vessel having an ostium comprising:
a balloon having a distal end and a long axis, a coil being disposed on the long axis;
means for advancing the distal end into the branch vessel;
means for inflating the balloon to form an expanded portion having a contacting edge;
means for deducing the ostium from the contacting edge;
means for determining whether the coil is within a desired distance of the ostium; and
means for recording a location of the coil when the coil is within the desired distance of the ostium.

19. The system of claim 18 further comprising:
means for deflating the balloon when the coil is not within the desired distance of the ostium; and
means for positioning the coil at the ostium.

20. The system of claim 18 further comprising means for correlating the location of the coil and an indicated ostium on a fluoroscopic image.

21. The system of claim 18 further comprising:
means for deflating the balloon;
means for advancing the distal end further into the branch vessel;
means for inflating the balloon; and
means for recording a second location of the coil to locate a branch vessel centerline.

22. The system of claim 18 further comprising means for recording a plurality of coil locations over a time period.

23. The method of claim 22 further comprising:
means for recording a body function over the time period; and
means for correlating the plurality of coil locations with the body function;

24. The method of claim 23 wherein the body function is selected from the group consisting of cardiac cycles and respiratory cycles.

25. The system of claim 18 wherein the coil is a first coil, and the balloon further comprises a second coil disposed on the long axis between the first coil and the distal end, further comprising:
means for recording a second coil location of the second coil to locate a branch vessel centerline.
